(19) United States
(12) Patent Application Publication
(10) Pub. No.: US 2026/0027067 A1
(43) Pub. Date: Jan. 29, 2026

(54) TRIENTINE LIQUID DOSAGE FORMS

(71) Applicant: BIOPHORE INDIA PHARMACEUTICALS PVT. LTD., Hyderabad (IN)

(72) Inventors: Manik Reddy PULLAGURLA, Hyderabad (IN); Jagadeesh Babu RANGISETTY, Hyderabad (IN); Srinivas ARUTLA, Hyderabad (IN); Praveen Kumar TALASILA, Hyderabad (IN); Srikanta PATRA, Hyderabad (IN)

(21) Appl. No.: 18/993,286

(22) PCT Filed: Aug. 23, 2023
(86) PCT No.: PCT/IB2023/058377

§ 371 Date: Jan. 10, 2025

(30) Foreign Application Priority Data
Aug. 27, 2022 (IN) 202241049001

(51) Int. Cl. A61K 31/132; A61K 9/00; A61K 9/08; A61K 47/02; A61K 47/18

(52) U.S. Cl. CPC A61K 31/132; A61K 9/0095; A61K 9/08; A61K 47/02; A61K 47/183

(57) ABSTRACT

A pharmaceutical liquid dosage form of Trientine and/or pharmaceutically acceptable salts thereof is provided, which are suitable for oral administration. A process of preparing the liquid dosage form and use for treatment of Wilson's disease and related diseases are also provided.

### CROSS-REFERENCE TO RELATED APPLICATIONS

This application claims priority to PCT Application No. PCT/IB2023/058377, having a filing date of Aug. 23, 2023, which is based on IN 202241049001, having a filing date of Aug. 27, 2022, the entire contents both of which are hereby incorporated by reference.

### FIELD OF TECHNOLOGY

The following relates to a pharmaceutical liquid dosage form of Trientine and/or pharmaceutically acceptable salts thereof, which are suitable for oral administration. The following also relates to process of preparing the liquid dosage form and use for treatment of Wilson's disease and related diseases.

### BACKGROUND

Wilson's disease is a genetic disorder in which excess copper builds up in the body. It occurs in about 1 in 30,000 people. Symptoms usually begin between the ages of 5 and 35 years. Symptoms are typically related to the brain and liver. Liver-related symptoms include vomiting, weakness, fluid build-up in the abdomen, swelling of the legs, yellowish skin, and itchiness. Brain-related symptoms include tremors, muscle stiffness, trouble in speaking, personality changes, anxiety, and psychosis. Penicillamine intolerant patients are treated with Trientine also known as triethylenetetramine, or 1,2-ethanediamine, N,N′-bis(2-aminoethyl) (TETA) has the structure:

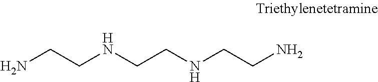

Various salts of Trientine including dihydrochloride salt (TETA·2HCl), tetrahydrochloride TETA·4HCl) is a polyamine chelator of copper (II). Its copper chelating properties make it useful in the treatment of various conditions, in particular Wilson's disease. The copper chelating ability of TETA also led to its consideration for the treatment of numerous conditions such as internal organ damage in diabetes patients, Alzheimer's disease and cancer.

Trientine and its salts are disclosed in EP01778618. It also discloses method of treating Wilson's disease. WO 2006/027705, U.S. Pat. Nos. 10,988,436B2, 11,072,577B2 disclose various novel forms of Trientine and its salts, their methods of preparation and treatment for Wilson's disease.

Trientine dihydrochloride salt (TETA·2HCl) is a white to pale yellow crystalline hygroscopic powder. It is freely soluble in water, soluble in methanol, slightly soluble in ethanol, and insoluble in chloroform and ether whereas Trientine tetrahydrochloride salt (TETA·4HCl) white, not hygroscopic crystalline powder, freely soluble in water.

Trientine was first approved for medical use in the United States in 1985. It is available as oral solid formulations like capsule and tablet. Specifically, Trientine dihydrochloride salt (TETA·2HCl) is approved as capsule dosage form, Trientine tetrahydrochloride salt (TETA·4HCl) is approved as tablet dosage form. Various approved strengths are 150 mg, 167 mg, 200 mg, 250 mg and 300 mg. The capsule dosage needs to be stored at 2° to 8° C. (36° to 46° F.). However, a solid formulation when need to be swallowed is always brings challenges to patients. Further these are unsuitable for patients who have difficulty swallowing, such as children, elderly patients, stroke patients, phagophobia patients, many other persons including disabled or incapacitated patients and/or the like. In order to overcome this problem, it would be beneficial to provide a Trientine containing composition as a liquid dosage form which is palatable, as well as physically and chemically stable.

### SUMMARY

An aspect relates to a Trientine or pharmaceutically acceptable salts thereof liquid dosage form. In one aspect, the Trientine liquid dosage form comprises Trientine and pharmaceutically acceptable salts thereof and a carrier.

In a further aspect, Trientine liquid dosage form contains Trientine as a pharmaceutically acceptable salt among Trientine dihydrochloride, Trientine tetrahydrochloride or a mixture thereof. In a further aspect aspect, Trientine liquid dosage form contains Trientine tetrahydrochloride.

In a further aspect aspect, Trientine liquid dosage form is a liquid or solid formulation for reconstitution into liquid. In a further aspect aspect, the solid formulation for reconstitution to liquid dosage form is among powder, granules, dispersible tablet, effervescent tablet or a mixture thereof.

In a further aspect aspect, Trientine liquid dosage form contains a carrier which is a mixture of one or more pharmaceutically acceptable excipient selected from the group comprising water, solvents, stabilisers, polymers, additives, antioxidants, microbial preservatives, buffering agents, aromatic agents, sweeteners, flavouring agent or diluents.

In a further aspect aspect, Trientine liquid dosage forms have Trientine concentration between about 10 mg/ml to 200 mg/ml, for example 20 mg/ml to 150 mg/ml, as a further example 25 mg/ml to 100 mg/ml of total the liquid dosage form.

In a further aspect aspect, Trientine liquid dosage forms in form of liquid have pH between about 2.0 to about 9.0, for example between about 2.0 to about 8, for example below 4.0, as a further example, between 2.0 to 3.5.

In a further aspect aspect, the Trientine liquid dosage form comprises Trientine or a pharmaceutically acceptable salt and one more of (i) a stabiliser, (ii) a flavouring agent (iii) a sweetener (iv) a buffer (v) a preservative and (vi) water; wherein the pH of the formulation is between about 2.0 and about 8.0.

In a further aspect aspect, Trientine liquid dosage forms are packed in any of the packs selected from the group comprising Pet bottles, HDPE containers, Amber Glass vials.

In a further aspect aspect, Trientine liquid dosage forms of embodiments of the present invention are palatable and stable.

In a further aspect aspect, the liquid dosage form comprising Trientine or a pharmaceutically acceptable salt is used for treatment of Wilson's disease.

### DETAILED DESCRIPTION

Provided herein are Trientine or a pharmaceutically acceptable salt liquid dosage forms. Also provided herein are Trientine or a pharmaceutically acceptable salt solid formulations for reconstitution into liquid dosage forms. The solid formulations for reconstitution are powder, granules, dispersible tablet, effervescent tablet and other solid forms which can be used to disperse or dissolve the solid content in a liquid carrier to prepare a liquid dosage form in a short period of time just before administration.

These Trientine formulations described herein are useful for the treatment of Wilson's Disease and related diseases. The formulations are advantageous over conventional solid dosage administration of Trientine ranging from ease of administration, accuracy of dosing, accessibility to additional patient populations such as to children and the elderly, and an increased patient compliance to medication. It is generally known that certain segments of the population have difficulty ingesting and swallowing solid oral dosage forms such as tablets and capsules. As many as a quarter of the total population has this difficulty. Often, this leads to non-compliance with the recommended medical therapy with the solid formulations, thereby resulting in rending the therapy ineffective. Further, solid formulations are not recommended for children or elderly due to increased risk in choking.

Furthermore, Trientine is approved in various markets as oral tablets and capsules for the treatment of patients with Wilson's disease who are intolerant of penicillamine. Trientine dihydrochloride is approved in the United States as 250 mg capsule and it is recommended initial dose of Syprine is 500-750 mg/day for paediatric patients and 750-1250 mg/day for adults given in divided doses two, three or four times daily. This may be increased to a maximum of 2000 mg/day for adults or 1500 mg/day for paediatric patients aged 12 or under.

Trientine dihydrochloride is approved in the United Kingdom as 200 mg capsule (300 mg Trientine dihydrochloride equivalent to 200 mg Trientine) and it is for the treatment of Wilson's disease in patients intolerant to D-Penicillamine therapy, in adults, adolescents and children aged 5 years or older; and the recommended dose is 800-1,600 mg (4-8 capsules) daily in 2 to 4 divided doses. Trientine tetrahydrochloride is approved in the United States as 300 mg tablet (300 mg of Trientine tetrahydrochloride equivalent to 150 mg Trientine) and its recommended starting total daily dosage in adult patients is 300 mg up to 3,000 mg taken orally in divided doses (two times daily). It further recommends avoiding use of the tablet in patients who are unable to swallow tablets. Likewise, Trientine tetrahydrochloride is approved in the United Kingdom as 150 mg tablet (equivalent to 150 mg Trientine) and its recommended dose is between 450 mg and 975 mg (3 to 6½ film-coated tablets) per day in 2 to 4 divided doses. Patient has to swallow Trientine tablets whole without splitting, crushing or chewing.

Current dosage Regimen suggest tittering dose of the therapy according to tolerance of the patient, weekly. Also dose adjustment is required in moderate to severe hepatic impairment. When the dose required is something other than the amount present in one or more intact solid formulations, the solid formulation must be divided to provide the correct dose. This leads to inaccurate dosing when solid dosages forms, such as tablets and capsule, are compounded to prepare other formulations for children.

In a further aspect aspect, Trientine liquid dosage forms described herein are prepared from the reconstitution of a Trientine solid formulation. In embodiments, the Trientine solid formulation comprising Trientine, a sweetener, a preservative, and optionally an excipient is dissolved in water, a buffer, other aqueous solvent, or a liquid to form a Trientine liquid dosage form.

In embodiments, the Trientine solid formulation herein further comprises a flavouring agent.

The solid formulations for reconstitution are powder, granules, dispersible tablet, effervescent tablet and other solid forms which can be used to disperse or dissolve the solid content in a liquid carrier to prepare a liquid dosage form in a short period of time just before administration.

In one aspect, the Trientine solid formulations described herein comprise Trientine or a pharmaceutically acceptable salt thereof and one or more pharmaceutically acceptable excipient. These solid formulations are prepared by the known methods.

Various amounts and concentrations of other components (sweeteners, buffers, preservatives, and the like) in the Trientine solid formulations are found in the previous section describing the amounts and concentrations for the analogous Trientine liquid dosage forms.

Liquid vehicles suitable for the Trientine solid formulations to be reconstituted into a liquid solution described herein are selected for a particular solution formulation (solution, suspension, etc.) as well as other qualities such as clarity, toxicity, viscosity, compatibility with excipients, chemical inertness, palatability, odour, colour and economy. Exemplary liquid vehicles include water, ethyl alcohol, glycerine, propylene glycol, syrup (sugar or other sweetener based, sugar-free flavoured syrup), juices (apple, grape, orange, cranberry, cherry, tomato and the like), other beverages (tea, coffee, soft drinks, milk and the like), oils (olive, soybean, corn, mineral, castor and the like), and combinations or mixtures thereof. Certain liquid vehicles, e.g., oil and water, can be combined together to form emulsions.

In embodiments, water is used for as a vehicle for a Trientine liquid dosage form. In embodiments, syrup is used for as a vehicle for a Trientine liquid dosage form. In embodiments, a juice is used as a vehicle for a Trientine liquid dosage form.

In embodiments, the Trientine liquid dosage forms prepared from the powder formulations described herein are homogenous. Homogenous liquids as used herein refer to those liquids that are uniform in appearance, identity, consistency and drug concentration per volume. Non-homogenous liquids include such liquids that have varied colouring, viscosity and/or aggregation of solid particulates, as well as non-uniform drug concentration in a given unit volume. Homogeneity in liquids is assessed by qualitative identification or appearance tests and/or quantitative HPLC testing or the like. The mixing methods and excipients described herein are selected to impart a homogenous quality to a resultant Trientine liquid dosage form. Mixing methods encompass any type of mixing that result in a homogenous Trientine liquid dosage form.

In embodiments, a quantity of a Trientine powder formulation is added to a liquid vehicle and then mixed by a stirring, shaking, swirling, agitation element or a combination thereof. In certain instances, a fraction of a Trientine powder formulation (i.e., one-half, one-third, one-fourth, etc.) is added to a liquid vehicle, mixed by stirring, shaking, swirling, agitation or a combination thereof, and the subsequent powder fraction(s) is added and mixed.

In embodiments, a liquid vehicle is added to a Trientine solid solution formulation in a container, for example, a bottle, vial, bag, beaker, syringe, or the like. The container is then mixed by stirring, shaking, swirling, agitation, inversion or a combination thereof. In certain instances, a fractional volume of the liquid vehicle (i.e., one-half, one-third, one-fourth volume, etc.) is added to a Trientine powder formulation in a container, mixed by stirring, shaking, swirling, agitation, inversion or a combination thereof; and the subsequent liquid fraction(s) is added and mixed. In certain instances, a one-half fractional volume of the liquid vehicle is added to a Trientine solid solution formulation in a container and mixing by shaking: the other one-half fractional volume of the liquid vehicle is then subsequently added and mixed.

For the Trientine solid formulations and liquid dosage forms described herein, kits and articles of manufacture are also described. Such kits can comprise a carrier, package, or container that is compartmentalized to receive one or more containers such as vials, tubes, and the like, each of the container(s) comprising one of the separate elements to be used in a method described herein including a Trientine powder or liquid dosage form. Suitable containers include, for example, bottles, vials, syringes, and test tubes. The containers can be formed from a variety of materials such as glass or plastic.

A kit typically may comprise one or more additional containers, each with one or more of various materials (such as reagents, optionally in concentrated form, and/or devices) desirable from a commercial and user standpoint for a Trientine powder or liquid dosage form described herein.

Non-limiting examples of such materials include, but not limited to, buffers, diluents, filters, needles, syringes, syringe adapter, carrier, package, container, vial and/or tube labels listing contents and/or instructions for use, and package inserts with instructions for use associated with a Trientine powder or liquid dosage form. A set of instructions will also typically be included.

In embodiments, liquid dosage forms include, but are not limited to, solutions (both aqueous and nonaqueous), suspensions, emulsions, syrups, slurries, juices, elixirs, dispersions, and the like. It is envisioned that solution/suspensions are also included where certain components described herein are in a solution while other components are in a suspension.

In one aspect, the Trientine liquid dosage forms described herein comprise Trientine and a carrier. The carrier is a mixture of one or more pharmaceutically acceptable excipient selected from the group comprising water, stabilisers, solvents, polymers, additives, antioxidants, microbial preservatives, buffering agents, aromatic agents, sweeteners, flavouring agent or diluents.

In embodiments, Trientine is present in about 10 mg/ml to about 200 mg/ml in the liquid dosage form or in the solid reconstituted liquid dosage form. In embodiments, Trientine is present in about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 135 mg/ml, about 140 mg/ml, about 150 mg/ml, about 155 mg/ml, about 160 mg/ml, about 170 mg/ml, about 180 mg/ml, about 190 mg/ml, about 195 mg/ml, or about 200 mg/ml in the liquid dosage form.

In embodiments, the Trientine liquid dosage form described herein comprises a solvent. A suitable solvent system comprises an acetone, methylenechloride, an alcohol or a mixture thereof. An alcohol is, for example, ethanol which may be denatured, for example, with butanone. Other suitable alcohols include, but are not limited to, methanol, propanol (e.g., isopropyl alcohol), butanol such as tert-butyl, etc., including mixtures thereof. Any suitable acetone may be used to carry out embodiments of the present invention, such as Pharmacopeial or USP grade acetone. Acetone and water may be used individually or combined as a solvent, and when combined may be used in any suitable ratio. In an embodiment, water alone without acetone is employed as the solvent. Other organic solvent systems encompass a wide variety of organic solvents (examples: tert butyl alcohol, ethanol, n-propanol, n-butanol, iso-propanol, ethyl acetate, acetone, methyl acetate, methanol, carbon tetrachloride, dimethylsulfoxide, chlorobutanol, cyclohexane, and acetic acid).

In embodiments, the Trientine liquid dosage form described herein comprises a sweetening agent. Sweeteners or sweetening agents include any compounds that provide a sweet taste. This includes natural and synthetic sugars, natural and artificial sweeteners, natural extracts and any material that initiates a sweet sensation in a subject. In embodiments, a solid/powder sweetener is used in the solution formulation described herein.

In embodiments, a liquid sweetener is used in the solution formulation described herein. Sugars illustratively include glucose, fructose, sucrose, xylitol, tagatose, sucralose, maltitol, isomaltose, hydrogenated isomaltose, lactitol, sorbitol, erythritol, trehalose, maltodextrin, polydextrose, and the like. Other sweeteners illustratively include glycerine, inulin, erythritol, maltol, acesulfame and salts thereof, e.g., acesulfame potassium, alitame, aspartame, neotame, sodium cyclamate, saccharin and salts thereof, e.g., saccharin sodium or saccharin calcium, neohesperidin dihydrochalcone, sativoside, thaumatin, and the like. Sweeteners can be used in the form of crude or refined products. Sweeteners can be used singly or in combinations of two or more. Suitable concentrations of different sweeteners can be selected based on published information, manufacturers' data sheets and by routine testing. In embodiments, the sweetening agent is sucralose. In embodiments, sucralose is present in about 0.5 mg/ml to about 3 mg/ml in the liquid dosage form. In embodiments, the sweetening agent is xylitol. In embodiments, xylitol is present in about 50 mg/ml to about 250 mg/ml in the solution formulation. In embodiments, the sweetener is saccharin. In some embodiments, saccharin is present in about 1 mg/ml to about 3 mg/ml in the solution formulation. In embodiments, the sweetening agent is sorbitol. In embodiments, sorbitol is present in about 50 mg/ml to about 250 mg/ml in the solution formulation.

In embodiments, the Trientine liquid dosage form described herein comprises a preservative. Preservatives include anti-microbials, antioxidants, and agents that enhance sterility. Exemplary preservatives include ascorbic acid, ascorbyl palmitate, tocopherols, butylated hydroxyanisole (BHA), butylated hydroxytoluene (BHT), citric acid, erythorbic acid, fumaric acid, malic acid, propyl gallate, tertiary butylhydroquinone, sodium ascorbate, sodium bisulfate, sodium metabisulfite, sodium sulfite, parabens (such as methylparaben, ethylparaben, propylparaben, butylparaben and their salts), benzoic acid, sodium benzoate, potassium sorbate, vanillin, and the like.

In embodiments, the preservative is a paraben. In embodiments, the paraben or mixture of parabens is present in about 0.1 mg/ml to about 2 mg/ml in the solution formulation. In embodiments, a paraben is present in about 0.01% w/w to about 5% w/w of the solid solution formulation. In embodiments, the preservative is sodium benzoate. In embodiments, sodium benzoate is present in about 0.5 mg/ml to about 1.2 mg/ml in the liquid dosage form. In embodiments, sodium benzoate is present in about 0.01% w/w to about 5% w/w of the solid solution formulation. In embodiments, the preservative is ascorbic acid. In embodiments, the ascorbic acid is present in about 0.1 mg/ml to about 2 mg/ml in the solution formulation.

In embodiments, the Trientine liquid dosage form described herein does not comprise a preservative. In further embodiments, the Trientine solution described herein does not comprise a paraben preservative. In further embodiments, the Trientine solution described herein does not comprise a paraben preservative when the formulation also comprises a sugar or sugar alcohol.

In an embodiment, the pH of the Trientine liquid dosage form or solid reconstituted liquid dosage form described herein is between about 2.0 to about 9.0.

In embodiments, the pH of the Trientine liquid dosage form or solid reconstituted liquid dosage form described herein is between about 2.0 to about 8.0.

In embodiments, the Trientine liquid dosage form described herein comprises a buffer. Buffering agents maintain the pH of the Trientine liquid dosage form described herein. Non-limiting examples of buffering agents include, but are not limited to sodium bicarbonate, potassium bicarbonate, magnesium hydroxide, magnesium lactate, magnesium glucomate, aluminium hydroxide, aluminium hydroxide/sodium bicarbonate co-precipitate, mixture of an amino acid and a buffer, a mixture of aluminium glycinate and a buffer, a mixture of an acid salt of an amino acid and a buffer, and a mixture of an alkali salt of an amino acid and a buffer. Additional buffering agents include citric acid, sodium citrate, sodium tartrate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts. Some buffering agents also impart effervescent qualities when a powder is reconstituted in a solution. In embodiments, the buffer concentration is between about 5 mM and about 50 mM.

In embodiments, modulation of the pH is desired to provide the lowest impurity profile. In embodiments, modulation of the pH is desired to provide increase the solubility of the solution.

In further embodiments, the Trientine liquid dosage form described herein comprises additional excipients including, but not limited to, glidants, flavouring agents, colouring agents and thickeners. Additional excipients such as bulking agents, tonicity agents and chelating agents are within the scope of the embodiments.

Glidants are substances that improve flowability of a powder. Suitable glidants include, but are not limited to, calcium phosphate tribasic, calcium silicate, cellulose (powdered), colloidal silicon dioxide, magnesium silicate, magnesium trisilicate, silicon dioxide, starch, talc and the like. In embodiments, the Trientine powder formulations for reconstitution to liquid dosage form described herein comprise a glidant.

In an embodiment, the Trientine liquid dosage form described herein comprises a flavouring agent or flavorant to enhance the taste or aroma of the formulation in liquid form. Suitable natural or synthetic flavouring agents can be selected from standard reference books, for example Fenaroli's Handbook of Flavour Ingredients, 3rd edition (1995). Non-limiting examples of suitable natural flavours, some of which can readily be simulated with synthetic agents or combinations thereof, include almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, grape, grapefruit, guava, hop, lemon, licorice, lime, malt, mandarin, molasses, nutmeg, mixed berry, orange, peach, pear, peppermint, pineapple, raspberry, rose, spearmint, strawberry, tangerine, tea, vanilla, wintergreen, etc. Also useful, particularly where the formulation is intended primarily for pediatric use, is tutti-frutti or bubblegum flavor, a compounded flavouring agent based on fruit flavours. Examples of desired flavoring agents include anise, cinnamon, cacao, orange, peppermint, cherry (in particular wild cherry), grape, bubblegum, vanilla, and mixed berry. Flavoring agents can be used singly or in combinations of two or more.

In further embodiments, the Trientine liquid dosage form described herein comprises a colouring agent for identity and/or aesthetic purposes. Suitable colouring agents illustratively include but not limited to FD&C Red No. 3, FD&C Red No. 20, FD&C Red No. 40, FD&C Yellow No. 6, FD&C Blue No. 2, D&C Green No. 5, D&C Orange No. 5, caramel, ferric oxide and mixtures thereof.

In further embodiments, the Trientine liquid dosage form described herein comprises a thickener. Thickeners impart viscosity or weight to the resultant liquid forms from the Trientine formulation described herein. Exemplary thickeners include but not limited to dextrin, cellulose derivatives (ethylcellulose, hydroxyethyl cellulose, methylcellulose, hypromellose, and the like) starches, pectin, polyethylene glycol, polyethylene oxide, trehalose and certain gums (xanthan gum, locust bean gum, etc.). In embodiments, the Trientine liquid dosage forms comprise a thickener.

Additional excipients are contemplated in the Trientine liquid dosage form embodiments. These additional excipients are selected based on function and compatibility with the Trientine liquid dosage forms described herein and may be found, for example in Remington: The Science and Practice of Pharmacy, Nineteenth Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999), herein incorporated by reference in their entirety.

In an embodiment, the sweetening agent is xylitol. In embodiments, the sweetening agent is sucralose.

Provided herein are various embodiments of Trientine liquid dosage forms. In one aspect, the Trientine liquid dosage form comprises Trientine or a pharmaceutically acceptable salt and one or more of (i) a stabiliser, (ii) a flavouring agent (iii) a sweetener (iv) a buffer (v) a preservative and (vi) water; wherein the pH of the formulation is between about 2.0 and about 8.0.

In an embodiment, Trientine liquid dosage form comprising of trientine tetrahydrochloride, sodium metabisulfite, disodium edetate, sucralose, orange flavor and purified water.

In an embodiment, preparation of the Trientine liquid dosage form described herein includes any known pharmaceutical method.

In an embodiment, the Trientine liquid dosage form described herein is prepared by dissolving the Trientine in a solvent, for example water or a mixture of water and an organic solvent, and adding the solution to a solution of buffer, the preservative, and the sweetener in water and adjusting the pH as needed with sodium hydroxide or hydrochloric acid. In an embodiment, the Trientine liquid dosage form described herein is prepared by dissolving the buffer, the preservative, Trientine, and the sweetener in water and adjusting the pH as needed with sodium hydroxide or hydrochloric acid. In an embodiment, the oral Trientine liquid dosage form described herein is prepared by dissolving citric acid anhydrous, sodium citrate anhydrous, sodium benzoate, Trientine, and xylitol in water and adjusting the pH as needed with sodium hydroxide or hydrochloric acid.

In embodiments, the order of addition does not matter.

In embodiments, the Trientine liquid dosage form described herein is prepared by the following steps:
a) dissolving sodium metabisulfite in water under continuous stirring;
b) adding other excipients disodium edeate, sucralose and orange flavour to step (a) under continuous stirring to get the clear solution;
c) adding Trientine tetrahydrochloride to step (b) under continuous stirring to get clear solution;
d) checking the pH of the solution;
e) optionally filtering the solution;
f) packing trientine tetrahydrochloride oral solution into amber glass bottles, optionally headspace purged with N₂ gas and closed with child resistant cap or closure.

The Trientine liquid dosage forms described herein are stable in various storage conditions including refrigerated, ambient and accelerated conditions. Stable as used herein refer to Trientine liquid dosage forms having about 95% or greater of the initial Trientine amount and about 5% w/w or less total impurities or related substances at the end of a given storage period. The percentage of impurities is calculated from the amount of impurities relative to the amount of Trientine. Stability is assessed by HPLC or any other known testing method.

In embodiments, the stable Trientine liquid dosage forms have about 5% w/w, about 4% w/w, about 3% w/w, about 2.5% w/w, about 2% w/w, about 1.5% w/w, about 1% w/w, or about 0.5% w/w total impurities or related substances.

In embodiments, the stable Trientine liquid dosage forms have about 5% w/w total impurities or related substances. In embodiments, the stable Trientine liquid dosage forms have about 4% w/w total impurities or related substances. In

In embodiments, the stable Trientine liquid dosage forms have about 3% w/w total impurities or related substances.

In embodiments, the stable Trientine liquid dosage forms have about 2% w/w total impurities or related substances. In embodiments, the stable Trientine liquid dosage forms have about 1% w/w total impurities or related substances.

At refrigerated and ambient conditions, the Trientine liquid dosage forms described herein are stable for at least 1 month, at least 2 months, at least 3 months, at least 6 months, at least 9 months, at least 12 months, at least 15 months, at least 18 months, at least 24 months, at least 30 months and at least 36 months. At accelerated conditions, the Trientine liquid dosage forms described herein are stable for at least 1 month, at least 2 months, at least 3 months, at least 4 months, at least 5 months, at least 6 months, at least 7 months, at least 8 months, at least 9 months, at least 10 months, at least 11 months or at least 12 months. Accelerated conditions include temperature and/or relative humidity (RH) that are above ambient levels (e.g., 25±0.5° C.: 55±10% RH).

In one aspect, the Trientine liquid dosage forms are stored at room temperature. In one aspect, the Trientine liquid dosage forms are stored at a controlled room temperature of 15° C. and 30° C. (59° F. and 86° F.), for example, at 20° C. to 25° C. (68° F. to 77° F.). In a further aspect, the Trientine liquid dosage forms are stored at 2° to 8° C. (36° to 46° F.).

In one aspect, the Trientine liquid dosage forms are used for the treatment of diseases and conditions described herein to a subject. In embodiments, the Trientine liquid dosage forms described herein treat Wilson's disease. In addition, a method for treating any of the diseases or conditions described herein in a subject in need of such treatment, involves administration of Trientine liquid dosage forms in therapeutically effective amounts to the subject.

Dosages of Trientine liquid dosage forms described can be determined by any suitable method. Maximum tolerated doses (MTD) and maximum response doses (MRD) for Trientine can be determined via established animal and human experimental protocols as well as in the examples described herein. For example, toxicity and therapeutic efficacy of Trientine can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Trientine dosages exhibiting high therapeutic indices are of interest. The data obtained from cell culture assays and animal studies can be used in formulating a range of dosage for use in human. The dosage of such compounds lies for example within a range of circulating concentrations that include the ED50 with minimal toxicity. The dosage may vary within this range depending upon the dosage form employed and the route of administration utilized.

In embodiments, the amount of a given Trientine liquid dosage form that corresponds to such an amount varies depending upon factors such as the particular Trientine salt or solvate, disease condition and its severity, the identity (e.g., weight, sex) of the subject or host in need of treatment, but can nevertheless be determined according to the particular circumstances surrounding the case, including, e.g., the specific agent being administered, the liquid dosage form type, the condition being treated, and the subject or host being treated.

In embodiments, the Trientine liquid dosage forms described herein are provided in a dose per day from about 50 mg to 5000 mg, from about 75 mg to about 4500 mg, from about 100 to about 4000, from about 125 mg to about 3500 mg of Trientine.

In embodiments, the Trientine liquid dosage forms described herein are provided in a daily dose of about 50 mg, about 75 mg, about 100 mg, about 125 mg, about 150 mg, about 175 mg, about 200 mg, about 225 mg, about 250 mg, about 275 mg, about 300 mg, about 350 mg, about 400 mg, about 450 mg, about 500 mg, about 600 mg, about 700 mg, about 800 mg, about 900 mg, about 1000 mg, about 1250 mg, about 1500 mg, about 1750 mg, about 2000 mg, about 2500 mg, about 3000 mg, about 3500 mg, about 4000 mg, about 4500 mg, or about 5000 mg, or any range derivable therein. The dose per day described herein can be given once per day or multiple times per day in the form of sub-doses given b.i.d., t.i.d., q.i.d., or the like where the number of sub-doses equal the dose per day. In further embodiments, the daily dosages appropriate for the Trientine liquid dosage forms described herein are from about 1.0 to about 20.0 mg/kg per body weight.

In further embodiments, the Trientine liquid dosage forms are provided in a dosage that is similar, comparable or equivalent to a dosage of a known Trientine tablet formulation. In embodiments, the Trientine liquid dosage forms are provided in a dosage that provides similar, comparable or equivalent pharmacokinetic parameters (e.g., AUC, Cmax, Tmax, Cmax, T1/2) as a dosage of a known Trientine tablet formulation. Similar, comparable or equivalent pharmacokinetic parameters, in some instances, refer to within 80% to 125%, 80% to 120%, 85% to 125%, 90% to 110%, or increments therein, of the given values. It should be recognized that the ranges can, but need not be symmetrical, e.g., 85% to 105%.

In an embodiment, there is provided a Trientine liquid dosage form wherein the mean plasma concentration of the Trientine after single dose administration exhibits a pharmacokinetic profile which is superior to that attainable by a product that is not a liquid dosage form.

In an embodiment, there is provided a method for providing a therapeutic blood plasma concentration of Trientine over a period of up to 24 hours resulting in improved pharmacological effect result which comprises administering orally to a patient in need thereof a Trientine liquid dosage form conducive to pharmacologically effective Trientine blood plasma levels from about half an hour to about 24 hours.

In an embodiment, the Trientine liquid dosage form provides a mean AUC of plasma Trientine in the subject which is greater than the mean AUC of plasma Trientine provided by a Trientine tablet, wherein the dose of Trientine in the solution formulation is the same as or less than the dose in the tablet dosage form.

In an embodiment, the mean AUC of plasma Trientine is about 10% to 50%, about 15% to about 30%, about 5% to 15%, about 5% to 20%, about 10% to 20%, about 10% to 25%, about 15% to 25%, about 20% to 25%, about 20% to 30%, about 20% to 40%, or about 30% to 40% greater than the AUC of plasma Trientine provided by administration of the tablet dosage form to the subject.

In an embodiment, the mean AUC of plasma Trientine is about 5%, 10%, 15%, 20%, 25%, 30%, 35%, 40%, 45%, 50% or more than about 50% greater than the mean AUC of plasma Trientine provided by administration of the tablet dosage form to the subject.

In an embodiment, the Trientine liquid dosage form shows a reduced “food effect” as compared to Trientine non-solution compositions. The compositions exhibit substantially similar oral bioavailability in fed and fasted subjects.

In an embodiment, an additional agent for use in combination with a Trientine liquid dosage form described herein. In an embodiment, Trientine liquid dosage form is free of another chelating agents. In an embodiment, Trientine liquid dosage form is free of Dimercaprol, Penicillamine, Zinc, Deferasirox, Deferiprone, Deferoxamine, Dimercaprol, EDTA and Succimer. In an embodiment, Trientine liquid dosage form is not administered with mineral supplements such as and not limited to iron, zinc, calcium, or magnesium.

In an embodiment, Trientine liquid dosage form is administered on an empty stomach (at least 1 hour before meals or 2 hours after meals and at least 1 hour apart from other food or milk). In an embodiment, concomitant use of mineral supplements (e.g. iron, zinc, calcium, magnesium) is avoided when Trientine liquid dosage form is administered. In an embodiment, when Trientine liquid dosage form is administered at least 2 hours before or 2 hours after taking an iron supplement at least 1 hour before or 2 hours after taking other mineral supplements.

In an embodiment, Trientine liquid dosage form is administered at least 1 hour apart from any other oral drug.

It will be apparent to those skilled in the art that various modifications and variations can be made in the methods and compositions of embodiments of the present inventions without departing from the spirit or scope of embodiments of the invention. Thus, it is intended that embodiments of the present invention cover the modification and variations of embodiments of the invention provided they come within the scope of the appended claims and their equivalents.

Unless defined otherwise, all technical and scientific terms used herein have the same meanings as commonly understood by one of ordinary skill in the art. Although any methods and materials similar or equivalent to those described herein can be used in the practice or testing of embodiments described herein, certain desired methods, devices, and materials are now described.

As used herein and in the appended claims, the singular forms “a”, “an”, and “the” include plural reference unless the context clearly dictates otherwise. Thus, for example, reference to “an excipient” is a reference to one or more excipients and equivalents thereof known to those skilled in the art, and so forth.

The term “about” is used to indicate that a value includes the standard level of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” The terms “comprise,” “have” and “include” are open-ended linking verbs. Any forms or tenses of one or more of these verbs, such as “comprises,” “comprising.” “has,” “having,” “includes” and “including,” are also open-ended. For example, any method that “comprises,” “has” or “includes” one or more steps is not limited to possessing only those one or more step and also covers other unlisted steps.

As used herein, “Trientine” refers to Trientine base, its salt, or solvate or derivative or isomer or polymorph thereof. Suitable compounds include the free base, the organic and inorganic salts, isomers, isomer salts, solvates, polymorphs, complexes etc. In embodiments, the Trientine used in the formulations described herein is a Trientine salt. In some instances, the Trientine used in the formulations described herein is a Trientine hydrochloride. In some instances, the Trientine used in the formulations described herein is Trientine dihydrochloride. In some instances, the Trientine used in the formulations described herein is Trientine tetrahydrochloride.

As used herein, “stable” refers to physical, chemical and polymorphic stability of Trientine base or its salt in embodiments of the invention liquid dosage forms. Physical stability refers to the physical characteristics of embodiments of the invention liquid dosage forms like appearance, description, colour, odour, taste, consistency and others. Chemical stability refers to the compliance of Assay and related substances values of Trientine base or its salt in line with the finished product specifications of embodiments of the invention liquid dosage forms, for example embodiments of the invention liquid dosage forms are stable at least for 6 months in 40° C./75% RH. Polymorphic stability refers to the retention of input API polymorphic form (Trientine base or its salt) throughout the finished product shelf-life, for example embodiments of the invention liquid dosage forms may be ready-to-use or the ones which may be reconstituted from solid formulations are comprising of Form A of Trientine tetrahydrochloride as described under U.S. Pat. No. 10,988,436.

As used herein, “adducts” refer to the formation of complexes of Trientine base or its salt with minute concentrations of detected elements in any component used in embodiments of the present invention liquid dosage form. The adducts are analyzed under the related substance testing.

While embodiments of the present invention have been shown and described herein, it will be obvious to those skilled in the art that such embodiments are provided by way of example only. Numerous variations, changes, and substitutions will now occur to those skilled in the art without departing from embodiments of the invention. It should be understood that various alternatives to the embodiments of the invention described herein may be employed in practicing embodiments of the invention. It is intended that the following claims define the scope of embodiments of the invention and that methods and structures within the scope of these claims and their equivalents be covered thereby.

### EXAMPLES

The following examples are provided to illustrate embodiments of the present invention. It should be understood, however, that embodiments of the invention is not to be limited to the specific conditions or details described in these examples.

Examples 1-4: Trientine tetrahydrochloride oral solution 60 mg/ml

| | Quantity in mg/ml | | | |
|---|---|---|---|---|
| Ingredients | Example 1 | Example 2 | Example 3 | Example 4 |
| Trientine Tetra | 60 | 60 | 60 | 60 |
| Hydrochloride | | | | |
| Purified Water | QS | QS | QS | QS |
| Sorbitol | — | 50 | 50 | 50 |
| Strawberry Flavor | — | 0.5 | 0.5 | 0.5 |
| Disodium hydrogen | QS for pH | QS for pH | QS for pH | QS for pH |
| phosphate | Adjust- | Adjust- | Adjust- | Adjust- |
| | ment | ment | ment | ment |
| Sodium citrate | QS for pH | QS for pH | QS for pH | QS for pH |
| | Adjust- | Adjust- | Adjust- | Adjust- |
| | ment | ment | ment | ment |
| pH of the Liquid | 2.5 | 2.53 | 6.00 | 3.2 |
| Dosage Form | | | | |

QS—Quantity sufficient

### Process of Preparation:

A clear solution obtained by adding Trientine tetrahydrochloride to purified water under continuous stirring.
Sorbitol and strawberry flavor were added into step no.1 under continuous stirring until a clear solution
Above solution was filtered through 0.22 μm membrane filter.
Filtered final solution was filled into amber coloured glass bottles.

Trientine tetra hydrochloride oral solutions of examples 1, 2, 3 & 4 were subjected to long term (25° C./60% RH) & accelerated stability (40°/75% RH). Drug Assay and Related substances (RS) were tested, and results represented in below tables.

| Accelerated | Example 1 | | | | Example 2 | | | |
|---|---|---|---|---|---|---|---|---|
| stability | Time points | | | | | | | |
| (40°/75% RH) | Initial | 1 M | 2 M | 6 M | Initial | 1 M | 2 M | 6 M |
| Impurity-A | ND | ND | 0.003 | ND | ND | ND | 0.008 | ND |
| Impurity-B | 0.130 | 0.130 | 0.136 | 0.250 | 0.120 | 0.130 | 0.144 | 0.280 |
| Highest Max | 0.03 | 0.09 | 0.026 | 0.099 | 0.05 | 0.05 | 0.049 | 0.103 |
| Unknown | | | | | | | | |
| Total | 0.23 | 0.38 | 0.281 | 0.530 | 0.330 | 0.320 | 0.345 | 0.690 |
| Impurities | | | | | | | | |
| Assay | 99.6 | 97.2 | 95.8 | 97.0 | 97.4 | 95.1 | 91.0 | 92.7 |

| Accelerated | Example 3 | | | | Example 4 | | | |
|---|---|---|---|---|---|---|---|---|
| stability | Time points | | | | | | | |
| (40°/75% RH) | Initial | 1 M | 2 M | 6 M | Initial | 1 M | 2 M | 6 M |
| Impurity-A | ND | ND | 0.252 | 1.886 | ND | ND | 0.01 | 0.122 |
| Impurity-B | 0.230 | 0.22 | 0.343 | 0.401 | 0.010 | 0.14 | 0.171 | 0.361 |
| Highest Max | 0.17 | 0.16 | 0.308 | 4.598 | 0.03 | 0.15 | 0.130 | 1.787 |
| Unknown | | | | | | | | |
| Total | 0.53 | 0.59 | 1.301 | 7.767 | 0.120 | 0.80 | 0.553 | 3.22 |
| Impurities | | | | | | | | |
| Assay | 83.8 | 78.8 | 92.1 | 94.3 | 100.9 | 99.0 | 95.1 | 88.7 |

| | Example 1 | | | Example 2 | | | Example 3 | | | Example 4 | | |
|---|---|---|---|---|---|---|---|---|---|---|---|---|
| Long term | Time points | | | | | | | | | | | |
| (25° C./60% RH) | Initial | 6 M | 9 M | Initial | 6 M | 9 M | Initial | 6 M | 9 M | Initial | 6 M | 9 M |
| Impurity-A | ND | ND | 0.003 | ND | ND | 0.019 | ND | ND | 0.123 | ND | 0.014 | 0.017 |
| Impurity-B | 0.130 | 0.285 | 0.134 | 0.120 | 0.255 | 0.142 | 0.230 | 0.541 | 0.402 | 0.010 | 0.292 | 0.165 |
| Highest Max | 0.03 | 0.102 | 0.027 | 0.05 | 0.105 | 0.058 | 0.17 | 0.331 | 0.311 | 0.03 | 0.225 | 0.386 |
| Unknown | | | | | | | | | | | | |
| Total | 0.23 | 0.546 | 0.307 | 0.330 | 0.606 | 0.481 | 0.53 | 7.767 | 1.626 | 0.120 | 0.917 | 1.073 |
| Impurities | | | | | | | | | | | | |
| Assay | 99.6 | 94.0 | | 97.4 | 93.3 | | 83.8 | 94.0 | | 100.9 | 87.9 | |

Impurity A: 1-(2-aminoethyl) piperazine Impurity. Specification limit is not more than 0.15%.

Impurity B: Diethylenetriamine Impurity. Specification limit is not more than 0.15%.

ND: Not detected.

Based on the aforementioned study results on trientine tetra hydrochloride oral solution 60 mg/mL it was concluded that, in terms of Assay and RS values of example 3 formulation was not good as it was exceeding the RS specification limits.

Further, based on an initial taste evaluation study carried out to understand the palatability of the formulation, it was noticed that example 1 is unpalatable. While example 2 and 4 were better when compared to example 1 but still there was a scope of improvement in taste or palatability.

Selection of sweeteners: Therefore, several trials were taken to optimize the stability and palatability of the formulation. New trials were conducted wherein a list of sweeteners were evaluated for their ability to mask the astringent taste of the API. Sweeteners such as sucralose, mannitol, saccharin sodium, etc., were evaluated. The ability of these sweeteners to mask the astringent taste of API was further evaluated at different sweetener concentrations. When saccharin sodium was used as sweetener, an incompatibility in the form of milky white curdling was observed which was an unexpected finding. Which led to saccharin sodium being eliminated from further trials. Sucralose emerged to be the best sweetener among the tested sweeteners. Further trials were carried out with sucralose as sweetener.

Selection of flavoring agents: A host of flavoring agents were also employed to determine their ability to enhance the palatability of the formulation. Flavors such as orange, lemon, grape, peppermint, strawberry, etc. were evaluated. Among the evaluated flavoring agents, orange flavor and strawberry flavor were imparting the best flavor to the formulations. Further trials were carried out with orange and strawberry flavor.

Selection of preservatives: Trials were taken with both antioxidant and chelating agent being in the formula and with either antioxidant or chelating agent being present in the formula. It was concluded that incorporation of both antioxidant and chelating agent would be beneficial to achieve stability of the formulation.

Selection of packing material: The evaluation of primary packaging was carried out simultaneously with optimizing the formula for the formulation. Packaging such as Pet bottles. HDPE containers. Amber Glass vials and Unit dose volume cups were evaluated.

PET bottles and Unit dose volume cups were showcasing wide ranging fluctuations in the formulations related substances limits because of which PET bottles and unit dose volume were disregarded as potential primary packaging materials. Additionally, the broad mouth of the unit dose volume cups was posing as a challenge at the time of induction sealing and Nitrogen (N₂) purging on the headspace.

HDPE containers or Glass vials were considered for primary packaging materials as they seemed to be compatible with the formulation. Between them. Glass vials were concluded as best suited for the formulation.

Example-5: The final composition was fixed based on the observations and inferences obtained from aforementioned trials.

| S. No | Name of ingredient | (mg/mL) | Category |
|---|---|---|---|
| 1 | Trientine tetrahydrochloride | (60) | API |
| 2 | Sodium metabisulfite | (0.5) | Antioxidant |
| 3 | Disodium Edeate | (0.5) | Chelating agent |
| 4 | Sucralose | (2.5) | Sweetener |
| 5 | Orange flavor | (0.5) | Flavoring agent |
| 6 | Purified water | QS | Vehicle |

QS—Quantity suffcient

### Process of Preparation:

A clear solution was prepared by adding Sodium metabisulfite to purified water under continuous stirring.
A clear solution was prepared by adding Disodium edetate to step-1 solution under continuous stirring.
A clear solution was prepared by adding Sucralose to step-2 solution under continuous stirring.
A clear solution was prepared by adding Orange flavour to step-3 solution under continuous stirring.
A clear solution was prepared by adding Trientine tetrahydrochloride to step-4 solution under continuous stirring. Final volume was adjusted with purified water.
pH of the solution was about 2.0 and the solution was filtered through 47 mm online filtration assembly.
The batch quantity of trientine tetrahydrochloride oral solution formulated was then packed into amber glass bottles or HDPE containers, headspace was purged with Nitrogen gas and white child resistant cap was used as closure.

Storage conditions: It was concluded that formulation can be stored at 20° C. to 25° C.; extrusions are permitted between 15° C. to 30° C. with a tightly closed cap.

Trientine tetra hydrochloride oral solutions of example 5 was subjected to accelerated stability (40°/75% RH). Drug Assay and Related substances (RS) were tested, and results represented in below table were well within the specification limits at least for 6 months of storage at 40°/75% RH.

| | Example-5 formulation | | | | | |
|---|---|---|---|---|---|---|
| | Packaging | | | | | |
| Accelerated | A - Amber glass vial | | | B - HDPE container | | |
| stability | Time (month) | | | | | |
| (40°/75% RH) | Initial | 3 months | 6 months | Initial | 3 months | 6 months |
| pH | 2.079 | 2.062 | 2.203 | 2.233 | 2.337 | 2.22 |
| Impurity-A | ND | 0.008 | ND | 0.003 | 0.008 | ND |
| Impurity-B | 0.026 | 0.041 | 0.033 | 0.027 | 0.041 | 0.032 |
| Highest | 0.073 | 0.082 | 0.061 | 0.027 | 0.040 | 0.039 |
| Maximum | | | | | | |
| Unknown | | | | | | |
| Total Impurities | 0.198 | 0.24 | 0.264 | 0.122 | 0.184 | 0.132 |
| Assay | 99.7 | 101.4 | 100.2 | 101.1 | 100.8 | 100.5 |

Comparative analytical results on identification of adducts: Trientine tetrahydrochloride being a copper chelator might inadvertently form chelate complexes or adducts with potential metallic molecules present in the excipients which are used in the formulation. To evaluate this, the example-5 oral solution was submitted for elemental impurities analysis according to ICH Q3D(R1) guidelines. The formulation was evaluated to determine the presence of all 24 listed elements in the guideline. The results of which are furnished in below table:

| S. No | Element | Result |
|---|---|---|
| 1 | Lithium (Li) | ND |
| 2 | Vanadium (V) | ND |
| 3 | Cobalt (Co) | ND |
| 4 | Arsenic (As) | ND |
| 5 | Molybdenum (Mo) | ND |
| 6 | Ruthenium (Ru) | ND |
| 7 | Rhodium (Rh) | ND |
| 8 | Palladium (Pd) | ND |
| 9 | Silver (Ag) | ND |
| 10 | Cadmium (Cd) | ND |
| 11 | Antimony (Sb) | ND |
| 12 | Osmium (Os) | ND |
| 13 | Iridium (Ir) | ND |
| 14 | Platinum (Pt) | ND |
| 15 | Mercury (Hg) | ND |
| 16 | Thallium (Tl) | ND |
| 17 | Lead (Pb) | 0.01 PPM |
| 18 | Chromium (Cr) | 0.01 PPM |
| 19 | Nickel (Ni) | 0.01 PPM |
| 20 | Selenium (Se) | 0.01 PPM |
| 21 | Gold (Au) | 0.03 PPM |
| 22 | Tin (Sn) | 0.05 PPM |
| 23 | Copper (Cu) | 0.07 PPM |
| 24 | Barium (Ba) | 0.09 PPM |

ND: Not detected.
NMT: Not more than.

From the results it was concluded that of the 24 elements tested, 8 elements are detected in detectable limits which were all well within the specified limits as seen in the specification. Based on the above findings, the formulation complies with Q3D(R1) guidelines. The inference is that minute concentrations of detected elements would not lead to the formation of adducts with Trientine tetrahydrochloride, which was also justified under the related substance testing.

Although the invention has been illustrated and described in greater detail with reference to the exemplary embodiments, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

We claim:

1. A stable liquid dosage form comprising Trientine or a pharmaceutically acceptable salt thereof and one or more pharmaceutically acceptable excipients.

2. The stable liquid dosage form as claimed in claim 1, wherein the Trientine is selected from Trientine dihydrochloride, Trientine tetrahydrochloride or a mixture thereof.

3. The stable liquid dosage form as claimed in claim 1, wherein the one or more pharmaceutically acceptable excipients is selected from the group comprising of solvents, buffering agents, chelating agents, pH adjusting agents, antioxidants, microbial preservatives, sweeteners and flavouring agents.

4. The stable liquid dosage form as claimed in claim 1, wherein the stable liquid dosage form is reconstituted from a solid formulation selected from the group consisting of powder, granules, dispersible tablet or effervescent tablet.

5. A stable and palatable liquid dosage form comprising Trientine or a pharmaceutically acceptable salt thereof and at least one or more pharmaceutically acceptable excipients, wherein the liquid dosage form is substantially free of adducts.

6. A stable liquid dosage form comprising trientine tetrahydrochloride, sodium metabisulfite, disodium edetate, sucralose, orange flavor and purified water.

7. The stable liquid dosage form as claimed in claim 6, wherein the stable liquid dosage form comprises Trientine tetrahydrochloride at a concentration of between about 10 mg/ml to 200 mg/ml.

8. The stable liquid dosage form as claimed in claim 6, wherein the pH of the liquid dosage form is between about 2.0 to about 8.0.

9. A process of preparing the stable liquid dosage form of trientine tetrahydrochloride as claimed in claim 6 comprising: a) dissolving sodium metabisulfite in water; b) adding disodium edeate, sucralose and orange flavour to step (a) to get clear solution; c) adding Trientine tetrahydrochloride to step (b) to get clear solution; d) checking the pH of the solution; e) optionally filtering the solution; and f) packing trientine tetrahydrochloride oral solution into amber glass or HDPE bottles, optionally headspace purged with nitrogen gas and closed with child resistant cap or closure.

10. The stable liquid dosage form as claimed in claim 1, wherein the stable liquid dosage form is used for treatment of Wilson's disease.
